(19) United States
(12) Patent Application Publication
(10) Pub. No.: US 2021/0346572 A1
(43) Pub. Date: Nov. 11, 2021

(54) METHODS AND DEVICES FOR REPAIR OF CRUSHED PERIPHERAL NERVE INJURIES WITH ERYTHROPOIETIN

(71) Applicant: STL Laboratories Inc., Franklin Lakes, NJ (US)

(72) Inventor: Shu-Tung Li, Wyckoff, NJ (US)

(21) Appl. No.: 17/383,941

(22) Filed: Jul. 23, 2021

Related U.S. Application Data
(63) Continuation-in-part of application No. 16/280,424, filed on Feb. 20, 2019, now Pat. No. 11,116,876.
(60) Provisional application No. 62/632,619, filed on Feb. 20, 2018.

(51) Int. Cl. A61L 27/22; A61K 38/18; A61L 27/48

(52) U.S. Cl. CPC A61L 27/227; A61K 38/1816; A61L 2300/412; A61L 2430/32; A61L 2300/252; A61L 27/48

(57) ABSTRACT

An implantable drug-delivery device for repairing a crushed peripheral nerve. The drug-delivery device includes a matrix formed of a biopolymer and an erythropoietin (EPO) entrapped in the matrix. After in vivo implantation of the drug-delivery device, the EPO elutes over a period of 1 day to 12 weeks. Also disclosed is a method for repairing a crushed peripheral nerve using the implantable drug-delivery device.

### CROSS-REFERENCE TO RELATED APPLICATIONS

This application is a continuation-in-part of U.S. patent application Ser. No. 16/280,424 filed on Feb. 20, 2019, which claims the priority of U.S. Provisional Application 62/632,619, filed on Feb. 20, 2018.

### BACKGROUND

Peripheral nerve injuries affect a large population worldwide. Two types of injuries to peripheral nerves are commonly observed. One type of injury results in severing of the nerve in which axons are completely disconnected at the injury site and the nerve continuity must be restored for functional recovery. The other type of injury is a crush injury in which nerves are not severed but are traumatized to various extents. Clinical manifestations of the crushed nerve injury include a variable degree of motor and sensory deficit.

Spontaneous recovery of a mild crush injury is often observed. In the case of a moderate crush injury, time-dependent partial functional recovery is typical. A severe crush injury, on the other hand, results in complete loss of motor function and intractable neuropathic pain that often necessitates surgery to repair the nerve for the return of function.

The major clinical objective in the treatment of crushed nerve injury, particularly the moderate to severe crushed nerve, is to accelerate the return of motor and sensory function of the injured nerves.

Recent studies have shown that erythropoietin (“EPO”) has protective effects on Schwann cells and neurons, leading to restoration of myelination of the injured nerves and speedy recovery of nerve injuries. See, e.g., Fowler et al. 2015, J. Nature and Science, 1(8): e166; Zhang, et al., Biomed Res Int. 2015; 2015:478103; Sundem et al. 2016, J. Hand Surg. Am. 41:999-1010; Geary et al. 2017, Muscle Nerve 56:143-151; Modrak, et al., Neural Regen Res. 2017, 12: 1268-1273, and Yin, et al., 2018, Am. J. Neuroradiol. 31:509-15.

Methods and devices are needed to (i) protect cells in a crushed nerve from degeneration, particularly neurons and Schwan cells, (ii) improve and accelerate the myelination of partially injured but not severed nerves, (iii) increase the total number of myelinated axons across the injury, and (iv) improve the speed of myelination and the extent of functional recovery.

### SUMMARY

To meet the needs set forth above, an implantable drug-delivery device for the treatment of a crushed peripheral nerve is provided. The drug-delivery device includes a matrix formed of a biopolymer and an erythropoietin (“EPO”) entrapped in the matrix. After in vivo implantation of the drug-delivery device, the EPO elutes over a period of 1 day to 12 weeks.

Within the scope of the invention are drug-delivery devices that elute EPO over a period of 1 day to 7-days, 1 to 3-weeks, 3 to 6-weeks, and 6 to 12-weeks after implantation.

The matrix mentioned above is semipermeable and is formed of a biopolymer that can be chitosan, alginic acid, cellulose, elastin, fibrin, a glycosaminoglycan, gelatin, a collagen, or a mixture of these biopolymers.

The implantable drug-delivery device can be in the form of a tube, a tubular wrapping cuff, a sheet, a rod, a strip, an injectable powder, or a sponge.

Also disclosed is a method for repairing a crushed peripheral nerve injury. The method is carried out by providing an implantable drug-delivery device that includes a matrix formed of a biopolymer and an EPO entrapped in the matrix and implanting the drug-delivery device at the site of the crushed nerve. After implantation in vivo of the drug-delivery device, the EPO elutes over 1 day to 12 weeks.

The details of one or more embodiments of the invention are set forth in the description below. Other features, objects, and advantages of the invention will be apparent from the description and from the claims.

### DETAILED DESCRIPTION

Described in detail below is an implantable drug-delivery device for repairing a crushed peripheral nerve that includes a matrix formed of a biopolymer.

The matrix is semi-permeable and is formed of a biopolymer that can be, but is not limited to, chitosan, alginic acid, cellulose, elastin, fibrin, a glycosaminoglycan, gelatin, or a collagen. The matrix can be formed of a mixture of these biopolymers.

The collagen is a fiber-forming collagen, e.g., collagen type I, type II, or type III, having a native structure. In other words, the device is free of denatured or cleaved collagens. In a particular device, the collagen is type I collagen.

The implantable drug-delivery device also includes an EPO entrapped in the matrix. The EPO can be purified natural EPO, recombinant EPO produced in bacteria or mammalian cells, or an EPO mimetic. See, e.g., U.S. Pat. No. 6,489,293.

As mentioned above, after in vivo implantation, the EPO elutes from the device over a period of 1 day to 12-weeks. For example, the EPO can elute over a 1 day to 7-day period, a 1 to 3-week period, a 3 to 6-week period, and a 6 to 12-week period. Any intermediate elution time between 1 and 12 weeks falls within the scope of the present invention.

Alternatively, the EPO can elute on a short-term basis, i.e., on the order of hours and even on the order of days. For example, a device is disclosed in which, following in vivo implantation, the EPO elutes over 1, 2, 4, 8, 12, 16, and 24 hours or over 2, 3, 4, 5, 6, and 7 days.

The EPO elution time can be selected for a particular application. For example, for repairing a mild crush injury, a device can be used that elutes EPO over a 1 to 7 day period after in vivo implantation. For a moderate crush injury, the device can elute EPO over 1 to 14 days, resulting in accelerated axon sprouting from the proximal healthy nerve end and protection of the partially injured axons from degeneration, thus facilitating crush wound healing and functional recovery.

The device can be constructed in several shapes, depending upon the application. The shapes include, but are not limited to, a flat membrane, a strip, a block, a rod, a thin filament, and a tubular wrapping cuff, i.e., a tube cut open along its long axis.

In a particular example, the device is a tubular nerve cuff device that contains EPO entrapped within its walls. Such a device serves as a nerve guide, a drug-delivery device, and a protective sheath for the injured nerve to block invasion of fibrogenic cells that deposit scar tissue.

In another embodiment, the device is a sheet strip or a rod device. In this embodiment, the device contains a porous collagen matrix and an EPO entrapped in the strip or rod matrix. The device can be inserted into the injured nerve via a small diameter cannula and released adjacent to the crushed nerve for EPO elution.

In yet another embodiment, the device is an injectable suspension of collagen powder matrix in a saline solution containing EPO within the collagen matrix. The collagen powder containing EPO can be delivered by needle injection at the injury site for EPO elution.

As also mentioned above, a method for repairing a crushed nerve is provided that includes a step of providing an implantable drug-delivery device that includes a matrix formed of a biopolymer and an EPO entrapped in the matrix. The device can be the same device described above. To reiterate, it can be a semi-permeable matrix formed of a biopolymer, e.g., chitosan, alginic acid, cellulose, elastin, fibrin, a glycosaminoglycan, gelatin, a collagen, and a mixture thereof. The collagen is a fiber-forming collagen, e.g., collagen type I, type II, or type III, having a native structure. The device is thus free of denatured or cleaved collagens. In a particular device, the collagen is type I collagen. The EPO entrapped in the matrix can be purified natural EPO, recombinant EPO produced in bacteria or mammalian cells, or an EPO mimetic, as set forth, supra.

When the crush injury is either mild or moderate, a strip EPO-collagen matrix implant can be inserted with a thin canulae along the injury site and the wound closed with a suture. This facilitates the release of EPO for promoting axonal growth, myelination, and return of function.

Alternatively, a powder form of an EPO-collagen matrix can be dispersed in water or saline and injected at the injury site to release EPO, resulting in a direct effect on nerve growth and myelination for faster return of function. In another alternative for treating moderate to severe crush injury, an EPO-collagen cuff implant can be surgically implanted around the injury site to release EPO, thereby protecting the injury site from the invasion of fibrogenic cells and reducing scar formation.

In certain severely crushed nerves, axons are damaged to such an extent that an extensive scar forms at the injury site which blocks the passage of newly generated axons through the scar to reach the distal nerve end. In this situation, the scar formed must be surgically removed and nerve continuity must be restored with a nerve guiding device along with the implantation of EPO-collagen device as described in U.S. patent application Ser. No. 16/280,424, U.S. Pat. No. 6,716,225, and US Patent Application Publication 2013/0345729.

After implantation in vivo of the drug-delivery device, the EPO elutes from the device over a period of 1 day to 12 weeks. In an exemplary method for treating a mild nerve crush injury, the EPO elutes from the device over 1 day to 7 days. For a moderate crush injury, the EPO elutes over 7 days to 14 days. In another example, if the injury is between moderate and severe, a device can be used that elutes EPO over 2 to 6 weeks. In a further example, if the injury is severe to the extent that treatment requires both removing the nerve scar and employing a nerve guide to bridge the gap beyond the critical size, a device that elutes EPO over 6-12 weeks can be used.

Without further elaboration, it is believed that one skilled in the art can, based on the disclosure herein, utilize the present disclosure to its fullest extent. The following specific examples are, therefore, to be construed as merely descriptive, and not limitative of the remainder of the disclosure in any way whatsoever. All publications and patent documents cited herein are incorporated by reference in their entireties.

### EXAMPLES

### Example 1: Preparation of Collagen Membranes

A type I collagen membrane was prepared from purified type I collagen fibers as follows. Bovine Achilles tendon tissue from 6-12-month-old animals were cleaned, frozen, and sliced into 0.5 mm thick slices. Purified type I collagen fibers were obtained from the slices by performing a series of extractions with water, acid, base, alcohol, and a salt solution to remove non-collagenous material from the tissue essentially as described in U.S. Pat. Nos. 6,955,524 and 8,821,917.

An aliquot of the purified type I collagen fibers was suspended in 0.7 M lactic acid, pH 2.5 overnight at 4° C. and subsequently homogenized to reduce the fiber size and achieve a uniform dispersion. The pH of the solution was then adjusted to the isoelectric point (˜pH 4.8) to reconstitute type I collagen fibers.

The reconstituted type I collagen fibers were partially dehydrated and laid on the surface of a flat polymer sheet, e.g., a polytetrafluoroethylene (PTFE) sheet. The collagen fibers were spread on the PTFE sheet by pressing them with a roller to form a matrix having a desired pore structure, thickness, and density.

More specifically, a fixed weight of partially dehydrated collagen fibers having a fixed amount of solid, i.e., collagen, per wet weight was compressed to a defined area and thickness, e.g., 0.1 mm to 2 mm, to achieve a defined wet matrix density. Upon freeze drying, a dry density in the range of 0.05 g/cm³ to about 0.5 g/cm³ was achieved. Within this density range the pore size is from about 100 μm to about 500 μm along the long axis of the pore. This pore size supports a permeability of macromolecules having a molecular weight of approximately 1,000,000 Dal or less.

The partially dehydrated membrane sheet formed as set out in the preceding paragraph was freeze-dried. The freeze-dried membrane sheet was subjected to chemical crosslinking using formaldehyde vapor to impart in vivo stability to the membrane.

Alternatively, the reconstituted type I collagen fibers mentioned, supra, were coated onto a rotating mandrel and partially dehydrated by pressing the fibers between a pair of glass plates. In this way, the thickness, density, and pore structure of the final implant were adjusted to suit its intended purpose.

The partially dehydrated tubular membrane was freeze-dried and then cut longitudinally prior to chemical crosslinking using formaldehyde vapor to impart in vivo stability to the membrane cross-linking.

### Example 2: Preparation of Tubular Collagen Cuff Matrix

A curled tubular cuff can provide uniform distribution of EPO around the proximal to distal regions of the crushed nerve injury and can facilitate the placement of the drug-delivery device to protect the injury site from scar formation. An exemplary device was prepared as set forth below.

Purified collagen fibers were suspended in 0.07 M lactic acid (pH 2.3) at a final collagen content of 0.7% (w/v) and swollen by incubation at 4° C. overnight. The swollen fibers were then homogenized to reduce the fiber size to fibrils to obtain a uniform dispersion. After de-gassing under vacuum, the pH of the dispersion was adjusted to the isoelectric point of collagen (˜pH 4.8) with 1M NH₄OH to reconstitute the dispersed collagen fibers.

The reconstituted collagen fibers were evenly wrapped around a rotating PTFE mandrel (OD 5.0 mm) to form a tubular membrane. Collagen fibers in the tubular membrane were partially dehydrated as described above in Example 1 using a thickness control gauge to form the tubular membrane with a fixed wall thickness (0.3 mm) to control its permeability.

The partially dehydrated tubular membrane was freeze-dried, removed from the mandrel, and subjected to chemical crosslinking. More specifically, crosslinking was performed with vapor from a 2% formaldehyde solution, followed by extensive washing with H₂O to remove any residual formaldehyde.

After washing, the tubular membrane was freeze-dried and cut along its longitudinal axis to form a curled tubular cuff.

### Example 3: Preparation of EPO-Collagen Composite Matrix Implants for Short-Term and Intermediate-Term In Vivo EPO Release

A fixed weight of partially dehydrated collagen fibers having a fixed amount of solid, i.e., collagen, per wet weight was compressed to a defined area and thickness, e.g., 0.1 mm to 2 mm, to achieve a defined wet matrix density. Upon freeze drying, a dry density in the range of 0.05 g/cm³ to about 0.5 g/cm³ was achieved. Within this density range the pore size is from about 100 μm to about 500 μm along the long axis of the pore. This pore size supports a permeability of macromolecules having a molecular weight of approximately 1,000,000 Dal or less.

The partially dehydrated membrane sheet formed as set out in the preceding paragraph was freeze-dried. The freeze-dried membrane sheet was subjected to chemical crosslinking using formaldehyde vapor to impart in vivo stability to the membrane.

Alternatively, the reconstituted type I collagen fibers mentioned, supra, were coated onto a rotating mandrel and partially dehydrated by pressing the fibers between a pair of glass plates. In this way, the thickness, density, and pore structure of the final implant were adjusted to suit its intended purpose.

The partially dehydrated tubular membrane was freeze-dried and then cut longitudinally prior to chemical crosslinking using formaldehyde vapor to impart in vivo stability to the membrane.

EPO was obtained from Bon Opus Biosciences (Summit, N.J.). The weight to bioactivity conversion was 1 ng of EPO to 1.2 IU. This relationship was determined as follows. A series of dilutions of EPO in saline was prepared over a concentration range of 1.5625 pg. to 100 pg. The amount of EPO, expressed as IU, in each dilution was determined with an EPO ELISA kit as directed by the manufacturer (ThermoFisher Scientific, Waltham, Mass.). The concentration of EPO in each sample by weight was equated to the concentration in IU.

EPO (60,000 IU) was dissolved in 0.5 ml of phosphate buffered saline (PBS; pH 7.2) to make a stock solution. Samples of the stock solution were diluted with PBS to make solutions containing 12 IU, 120 IU, 900 IU, and 1,200 IU of EPO in 100 μl. Each EPO solution was uniformly added via a volumetric micro-pipettor to a curled tubular collagen cuff delivery device.

A tubular collagen cuff was prepared as described in Example 2 above. The tubular cuff had an inside diameter of 5 mm and an outside diameter of 5.6 mm and a length of 20 mm. The pore sizes of the tubular collagen cuff were significantly larger than the size of EPO (M.W. 30.4 KDal, diameter ˜20 Å) to prevent surface adsorption of the EPO.

EPO was allowed to diffuse into the interstitial, i.e., intrafibrillar, space through the pores of the tubular collagen cuff to form an EPO-collagen composite matrix. Not to be bound by theory, it is believed that the EPO interacts with the collagen fibers of the cuff via physical, mechanical, and electrostatic interactions. The EPO-collagen composite matrix was then air dried and stored at 4° C. or lower.

### Example 4: Preparation of a Powdered Form of EPO-Collagen Composite Matrix

To produce a powdered form of the EPO-collagen composite matrix, an EPO-collagen composite matrix was produced as described above in Example 3.

The EPO-collagen composite matrix was air dried and pulverized in the presence of dry ice to particles having a size less than 100 μm to facilitate injection via a syringe. The powdered EPO-collagen composite matrix was also stored at 4° C. or lower.

### Example 5: Preparation of EPO-Collagen Composite Matrices for Long-Term Sustained Release of EPO In Vivo Up to 12 Weeks

To release EPO at a slow and sustainable rate, the EPO was entrapped within a collagen matrix during the reconstitution of collagen fibers as described above in Example 2. To accomplish this, a fixed amount of EPO (1000 IU to 5000 IU) was dissolved in PBS, pH 7.2. The EPO solution was then mixed with a 0.7% (w/v) collagen dispersion, pH 2.3, prior to reconstituting the collagen fibers by adjusting the pH to the isoelectric point of collagen (pH 4.8) with 1 M NH₄OH. Under these conditions, EPO was co-reconstituted together with collagen fibrils.

Further processing and engineering the reconstituted yet still hydrated EPO/collagen fibers into an EPO-collagen cuff matrix was performed as described above in Example 2.

The EPO-collagen cuff matrix was stabilized by formaldehyde vapor crosslinking to form a matrix that can be maintained for long periods of time in vivo. To avoid loss of EPO, residual formaldehyde was removed by venting for 72 to 96 h instead of by H₂O rinsing as in Example 2. The venting reduced the residual amount of formaldehyde to a level safe for in vivo implantation.

The desired in vivo stability of the EPO-collagen cuff matrix was controlled by the extent of formaldehyde crosslinking. The expected in vivo stability of the matrix was estimated by measuring hydrothermal shrinkage temperature of the matrix by differential scanning calorimetry.

The permeability of the collagen cuff matrix was adjusted during its formation to reduce the rate of diffusion of EPO to obtain sustained release over a longer period of time. For example, one way to reduce permeability is to increase the density of the engineered collagen cuff matrix. This was accomplished by more extensive dehydration of the reconstituted EPO-collagen cuff matrix prior to freeze-drying during the matrix engineering process.

Generally, the higher the density, the smaller the pore size, thus the slower the permeability, which in turn decreases the rate of EPO release.

At a density of 0.35-0.50 g/cm³, only about 50% of the interstitial space in the EPO-collagen cuff matrix is open. As a result, the movement of EPO will be significantly restricted and the physical, mechanical, and electrostatic interactions are enhanced and stabilized to reduce the rate of EPO release.

The length of the EPO-collagen cuff matrix was such that it would cover the length of a crushed nerve.

### Example 6: Determination of the Extent of EPO Incorporation

The incorporation efficiency of EPO for the short- and intermediate-term EPO-collagen matrix implants described above in Example 4 was 100%, as all of the EPO solution delivered via micropipette was absorbed into the matrix.

Turning to the sustained release EPO-collagen matrix implant of Example 5, EPO incorporation efficiency was determined by the weight difference between the total EPO added to the dispersion and the residual EPO left in the solution after the EPO-collagen matrix was reconstituted. EPO was measured by ELISA assay mentioned above in Example 4.

As a control, a length of collagen tubular cuff matrix was soaked in 1 ml of a solution of 550 IU/ml EPO, and the amount of EPO remaining in the solution measured by ELISA.

The results are shown in Table 1 below.

TABLE 1
Amount of EPO in Collagen Cuffs

| | | Micropipetting | Co-reconstituting |
|---|---|---|---|
| | Soaking | (Example 3) | (Example 5) |
| Initial EPO amount | 550 IU/ml | 12-1,200 IU/100 μl | 480 IU/5 ml |
| Absorbed EPO (IU) | 434 ± 11.6 | 12-1,200 | 463 |
| Efficiency of | 78.8 ± 02.1 | 100 | 96.03 |
| incorporation (%) | | | |

The efficiencies of EPO incorporation via micropipette and via co-reconstitution were similar, and both were higher than that achieved by soaking the collagen cuff matrix in an EPO solution.

### Example 7: Kinetics of EPO Release In Vitro

To determine the rate and quantity of EPO release, fixed weights (30 mg) of EPO-collagen composite matrices with known EPO quantity were incubated in 1 ml volumes of PBS (pH 7.2) at 37° C. with constant shaking. Aliquots of 10 μl of PBS containing EPO released from the matrix samples were collected and EPO content determined by ELISA as described above. The results are shown in Table 2 below.

TABLE 2
EPO release kinetics from collagen cuffs

| EPO | | | | | | | | | | |
|---|---|---|---|---|---|---|---|---|---|---|
| amount | | days | | | | | | | | |
| loaded | | 1 | 3 | 5 | 7 | 10 | 14 | 21 | 28 | 35 |
| 12 IU | amt. | 0.04^{a} | 0.07 | 0.07 | N.D.^{d} | N.D.^{d} | N.D.^{d} | N.D.^{d} | N.D.^{d} | N.D.^{d} |
| | released | | | | | | | | | |
| | cumulative | 0.04^{b} | 0.11 | 0.18 | N.D.^{d} | N.D.^{d} | N.D.^{d} | N.D.^{d} | N.D.^{d} | N.D.^{d} |
| | % | 0.33^{c} | 0.92 | 1.50 | N.D.^{d} | N.D.^{d} | N.D.^{d} | N.D.^{d} | N.D.^{d} | N.D.^{d} |
| 120 IU | amt. | 0.78 | 0.39 | 0.31 | 0.28 | 0.28 | 0.27 | 0.20 | N.D.^{d} | N.D.^{d} |
| | released | | | | | | | | | |
| | cumulative | 0.78 | 1.17 | 1.48 | 1.76 | 2.04 | 2.31 | 2.51 | N.D.^{d} | N.D.^{d} |
| | % | 0.65 | 0.98 | 1.23 | 1.47 | 1.70 | 1.93 | 2.09 | N.D.^{d} | N.D.^{d} |
| 900 IU | amt. | 22.6 | 29.4 | 10.1 | 5.0 | 3.0 | 3.2 | 3.8 | N.D.^{d} | N.D.^{d} |
| | released | | | | | | | | | |
| | cumulative | 22.6 | 52.0 | 62.1 | 67.1 | 70.1 | 73.3 | 77.1 | N.D.^{d} | N.D.^{d} |
| | % | 2.51 | 5.78 | 6.90 | 7.46 | 7.79 | 8.14 | 8.57 | N.D.^{d} | N.D.^{d} |
| 1200 IU | amt. | 89.0 | 51.0 | 21.6 | 9.2 | 18.2 | 14.4 | 6.9 | 7.5 | 4.7 |
| | released | | | | | | | | | |
| | cumulative | 89.0 | 140.0 | 161.6 | 170.8 | 189.0 | 203.4 | 210.3 | 217.8 | 222.5 |
| | % | 7.42 | 11.67 | 13.47 | 14.23 | 15.75 | 16.95 | 17.53 | 18.15 | 18.54 |
| 1200 IU | amt. | N.D.^{d} | N.D.^{d} | N.D.^{d} | N.D.^{d} | 3.7 | 3.9 | N.D.^{d} | N.D.^{d} | N.D.^{d} |
| sustained | released | | | | | | | | | |
| release | cumulative | N.D.^{d} | N.D.^{d} | N.D.^{d} | N.D.^{d} | 3.7 | 7.6 | N.D.^{d} | N.D.^{d} | N.D.^{d} |
| | % | N.D.^{d} | N.D.^{d} | N.D.^{d} | N.D.^{d} | 0.31 | 0.63 | N.D.^{d} | N.D.^{d} | N.D.^{d} |

^{a}amount of EPO (IU) eluted into PBS
^{b}cumulative amount of EPO (IU) released
^{c}cumulative amount of EPO released as percentage of initial amount loaded
^{d}N.D. = not determined

The rate of release of EPO from the short- and intermediate-term release EPO-collagen matrices was biphasic. Not to be bound by theory, it is expected that a pool of EPO in the matrices was physically and mechanically entrapped with collagen fibers and diffused out of the matrix at a faster rate, as compared to another pool of EPO more strongly associated with the collagen molecules or fibers via ionic bonds.

For particular in vivo applications, the initial fast-releasing pool of EPO can be removed from the EPO-collagen matrices prior to implantation, e.g., by incubating them in saline at 37° C. for 3-7 days depending on the initial amount of EPO incorporated.

In a particular example, incubating a 1200 IU EPO-collagen cuff matrix in saline for 7 days yielded a cuff that released EPO at a relatively constant rate over a two-week period. See data for the 1200 IU sustained release cuff listed in Table 2 above.

### Example 8: Local Delivery of EPO and its Effects on Nerve Regeneration

The efficacy of an EPO-collagen cuff matrix implant is tested in a rat sciatic nerve injury model. A crushed nerve injury is made at the moderate to severe level. The extent of injury is evaluated by correlating the histological observation with the extent of injury of the crushed nerve produced using a specially designed force sensor gauge device.

Briefly, sciatic nerves in control and experimental animals are crushed with the force sensor gauge device that can deliver quantitatively a force to the sciatic nerve per length for a defined period to cause local damage to the nerve.

The nerve injury is treated by wrapping a selected EPO-collagen cuff matrix implant around the injured nerve to release EPO locally and to protect the wound from scar formation.

Crushed nerves in the control group are treated by wrapping a collagen cuff matrix implant lacking EPO around the injured nerve.

Lewis rats are used, as this strain displays autophagia of the denervated limb less frequently than other rat species. See, e.g., Chamberlin, et al., 2000. A summary of the experiment is shown below in Table 3.

TABLE 3
Local EPO delivery animal study protocol summary

| | | Duration of | No. of animals | Analysis |
|---|---|---|---|---|
| | Group | implantation | per group | (histology) |
| Experimental | 120 IU | 7 days | 6 | Distal end of |
| | eluting cuff | 14 days | 6 | injury |
| Experimental | 480 IU | 7 days | 6 | Distal end of |
| | eluting cuff | 14 days | 6 | injury |
| Experimental | 900 IU | 7 days | 6 | Distal end of |
| | eluting cuff | 14 days | 6 | injury |
| Control | collagen cuff | 7 days | 6 | Distal end of |
| | without EPO | 14 days | 6 | injury |

In detail, 48 adult Lewis rats (˜200 g each) are anesthetized with an intraperitoneal injection of a mixture of ketamine HCl (90 mg/kg) and xylazine HCl (10 mg/kg), the hindquarters are shaved on the right side, scrubbed with betadine, and draped with a sterile towel while in the left side lying position. The right sciatic nerve of each animal is exposed through a longitudinal muscle splitting incision in the mid-thigh and dissected free from the underlying muscle bed.

The sciatic nerve is moderately crushed with the force sensor gauge device at the mid-thigh level with a length of 3 mm. In each experimental animal, one 5 mm length of fast-eluting EPO-collagen cuff matrix (120 IU, 480 IU, or 900 IU/rat—12 rats per dose) is implanted to wrap around the injured nerve segment. The inside diameter of the cuff is selected to loosely curl around the outside diameter of the injured nerve. A collagen cuff lacking EPO is implanted at the analogous site in 12 control rats. No suture is necessary to hold the cuff in place.

The muscle borders are approximated and sutured with 3-0 VICRYL™. The skin incision is closed with stainless steel staples. The dose range of EPO is selected based on the total EPO IU released for the first 7 days and 14 days based on in vitro release studies.

Animals are euthanized at day 7 and day 14 after surgery by intraperitoneal injection of 150 mg/kg pentobarbital. Rats are trans-cardially perfused with phosphate buffered saline (pH 7.2) followed by 4% paraformaldehyde in 0.1 M phosphate buffer (pH 7.2). After perfusion, the implanted nerve cuff including the injured nerve is dissected out, post-fixed in 1% glutaraldehyde and 2% paraformaldehyde in 0.1 M phosphate buffer for 24 h and treated for 1 h with 2% osmium tetroxide. Two micrometer transverse sections are cut at a defined distance from the end of the crushed nerve. The tissue slices are floated onto glass slides, stained with toluidine blue, mounted, and cover-slipped.

Partially overlapping light microscopy images of the entire cross section of the EPO-collagen cuff and sciatic nerve are captured using a digital camera and 40× objective and 100× oil immersion objective. The images are montaged using commercially available software and saved as one image of the entire section of the sciatic nerve. Every myelinated axon in the images is counted with Image J software. The number, diameter, and thickness of the myelin of myelinated axons are determined and compared between the experimental and control groups. The efficacy level of EPO dose is also determined.

In addition, blood vessels, mast cells, macrophages, and other cell types are identified and quantified. One equivalent section of the contralateral sciatic nerve is processed in the same manner for a normal control.

### Example 9 the Effect of EPO on Functional Recovery

Functional recovery after injured nerve treatment is assessed using a sciatic function index (“SFI”) as previously described by de Medinaceli et al. (1982, Exp. Neurology 77:634-643) and later adapted and modified by Chamberlain, et al. (2000, Neurosci. Res. 60:666-677).

Rats are subjected to nerve crush injury and treated with collagen cuffs with or without EPO as described in Example 8. The SFI is calculated based on the results of walking track measurements as previously described by Chamberlain, et al. More specifically, at predetermined time points post-surgery, the rat's hind paws are dipped into black non-toxic water-soluble ink. The rats then walk across a clean sheet of white paper that is placed in a walking chamber (10 cm wide×85 cm long) terminating in a dark box, leaving footprints from the hind limb. After several practice sessions, the rats walk straight to the end of the chamber to the dark box. Three footprint parameters are measured for analysis, i.e., (i) print length, (ii) toe spread, and (iii) intermediate toe spread. The initial measurements are modified according to Chamberlain et al. 2000 to calculate normalized footprint measurements (experimental value minus normal value divided by the normal value). The normalized values are designated as print length factor (“PLF”), toe spread factor (“TSF”) and intermediate toe spread factor (“ITF”). These normalized values are used to calculate SFI according to the following equation:

SFI=−38.3 (PLF)+109.5 (TSF)+13.3 (ITF)−8.8

The SFI is weighted so that normal function scores will be approximately −10 and no function scores are approximately −110. See Chamberlain, et al., 2000.

Following walking track assessment of functional recovery, animals are euthanized, and tissues processed as described in Example 8 above.

For tissue analysis, the injured nerve with collagen cuff is dissected, divided into proximal, middle, and distal segments of 1.5 mm in length, and processed for light microscopy as described above. One 4.5 mm section of the contralateral mid-thigh sciatic nerve is processed in the same manner for a normal control.

Partially overlapping images of the entire cross section of the nerve cuff injured sciatic nerve are captured and analyzed as set forth, supra.

### Example 10: Repair of Critically Crushed Nerve Injury that Requires Surgery for Scar Removal and the Use of EPO-Collagen Cuff

Treatment of a severely crushed nerve injury where the growth passage of the regenerated axons is blocked by scar tissue is as follows. First, the scar tissue is removed by surgery which results in a nerve gap. The repair of the severed nerve using a nerve conduit to guide the axonal growth through the nerve gap is known in the art and is described in the parent patent application, i.e., U.S. patent application Ser. No. 16/280,424. In situations where the gap length is beyond the critical size (about 2 cm in human and 1 cm in rat), a nerve conduit alone is not sufficient. The addition of an EPO-collagen cuff device is required as described in the parent application.

This experiment would provide guidance as to what level of injury requires a surgical intervention to treat the severely crushed nerve injury, i.e., by resecting the damaged nerve and repairing it using a nerve guide (entubulation) device in combination with the use of EPO-collagen cuff implant to enhance the nerve growth as described in the parent patent application.

The EPO-collagen cuff matrix described in Example 5 is used for this study. It is prepared initially with 900 IU of EPO incorporated into the collagen cuff matrix. Prior to implantation the cuff is rinsed for two days in saline to remove the fast-release pool of EPO, leaving only the sustained/slow-release pool. EPO-collagen cuff matrices are subjected to ethylene oxide sterilization prior to implantation. The releasable dose level of EPO is 1-5 IU/rat/day over a period of 1-12 weeks.

The rat sciatic nerve injury model discussed above is employed using a total of 36 animals (250 g-300 g each) divided into three groups as shown in Table 4 below.

TABLE 4
Severely crushed nerve injury experimental protocol

| | Time of | No. of |
|---|---|---|
| Group | implantation | animals |
| 1. Slow-release EPO | 6 weeks | 6 |
| cuff only | 12 weeks | 6 |
| 2. Fast-release EPO | 6 weeks | 6 |
| cuff (6 mm) at | 12 weeks | 6 |
| proximal end and a | | |
| slow-release EPO | | |
| cuff (8 mm) placed | | |
| next to the fast | | |
| release cuff | | |
| 3. control | 6 weeks | 6 |
| cuff only-no EPO | 12 weeks | 6 |

A 1.2 cm nerve gap (beyond the critical length in rats) is created upon the resection of 5 mm severely crushed nerve to remove the scar tissue. The nerve gap is first repaired with a neve guide of 1.4 cm. In Group 1, the slow-release EPO-collagen cuff matrix (14 mm) is implanted across the entire length of the nerve guide. In Group 2, a fast release EPO-collagen cuff matrix (6 mm length) is positioned at the proximal stump region and the slow-release EPO-collagen cuff matrix (8 mm length) is placed next to the fast release EPO-collagen cuff matrix, covering the middle section and the distal nerve stump. In control Group 3, a collagen cuff matrix lacking EPO is positioned across the length of the nerve guide. All groups are evaluated at 6 weeks and 12 weeks post-surgery.

Functional testing and histological examination are performed as described above in Examples 8 and 9.

### OTHER EMBODIMENTS

All of the features disclosed in this specification may be combined in any combination. Each feature disclosed in this specification may be replaced by an alternative feature serving the same, equivalent, or similar purpose. Unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. An implantable drug-delivery device for repairing a crushed peripheral nerve, the drug-delivery device comprising a matrix formed of a biopolymer and an erythropoietin (EPO) entrapped in the matrix, wherein, after in vivo implantation of the drug-delivery device, the EPO elutes over 1 day to 12 weeks.

2. The implantable drug-delivery device of claim 1, wherein the matrix is semipermeable, and the biopolymer is chitosan, alginic acid, cellulose, elastin, fibrin, a glycosaminoglycan, gelatin, a collagen, or a mixture thereof.

3. The implantable drug-delivery device of claim 2, wherein the biopolymer is collagen type I, type II, or type III.

4. The implantable drug-delivery device of claim 3, wherein the biopolymer is type I collagen.

5. The implantable drug-delivery device of claim 4, wherein the EPO elutes over 1 to 7 days.

6. The implantable drug-delivery device of claim 4, wherein the EPO elutes over 1 to 3 weeks.

7. The implantable drug-delivery device of claim 4, wherein the EPO elutes over 3 to 6 weeks.

8. The implantable drug-delivery device of claim 4, wherein the EPO elutes over 6 to 12 weeks.

9. The implantable drug-delivery device of claim 1, wherein the device is in the form of a tube, a tubular wrapping cuff, a sheet, a rod, a strip, an injectable powder, or a sponge.

10. A method for repairing a crushed peripheral nerve injury, the method comprising:
providing an implantable drug-delivery device that includes a matrix formed of a biopolymer and an erythropoietin (EPO) entrapped in the matrix, and
implanting the drug-delivery device at the site of the crushed nerve,
wherein, after implantation in vivo of the drug-delivery device, the EPO elutes over 1 day to 12 weeks.

11. The method of claim 10, wherein the matrix is semipermeable, and the biopolymer is chitosan, alginic acid, cellulose, elastin, fibrin, a glycosaminoglycan, gelatin, a collagen, or a mixture thereof.

12. The method of claim 11, wherein the biopolymer is collagen type I, type II, or type III.

13. The method of claim 12, wherein the biopolymer is type I collagen.

14. The method of claim 13, wherein the EPO elutes over 1 to 7 days.

15. The method of claim 13, wherein the EPO elutes over 1 to 3 weeks.

16. The method of claim 13, wherein the EPO elutes over 3 to 6 weeks.

17. The method of claim 13, wherein the EPO elutes over 6 to 12 weeks.

18. The method of claim 10, wherein the device is in the form of a tube, a tubular wrapping cuff, a sheet, a rod, a strip, an injectable powder, or a sponge.

19. The method of claim 18, wherein the drug-delivery device is affixed to an outer surface of the crushed nerve.

20. The method of claim 10, wherein the crushed nerve injury is mild and the EPO elutes over 1 to 7 days.

21. The method of claim 10, wherein the crushed nerve injury is moderate and the EPO elutes over 1 to 3 weeks.

22. The method of claim 10, wherein the crushed nerve injury is severe and the EPO elutes over 3 to 6 weeks.

23. The method of claim 10, further comprising resection of scar tissue and insertion of the crushed nerve into a nerve guide, wherein the crushed nerve is more than severely crushed and the EPO elutes over 6 to 12 weeks.
